(19) United States
(12) Patent Application Publication
(10) Pub. No.: US 2026/0018279 A1
(43) Pub. Date: Jan. 15, 2026

(54) SYSTEMS AND METHODS FOR RECOMMENDING DIAGNOSTIC ACTIONS FOR MEDICAL DEVICE DIAGNOSTIC TOOLS

(71) Applicant: KONINKLIJKE PHILIPS N.V., EINDHOVEN (NL)

(72) Inventors: Lu Wang, EINDHOVEN (NL); Milosh Stolikj, EINDHOVEN (NL); Qi Gao, EINDHOVEN (NL); Jurgen Jan Rusch, EINDHOVEN (NL); Emanuele Bastianelli, EINDHOVEN (NL)

(21) Appl. No.: 18/995,969

(22) PCT Filed: Jul. 10, 2023
(86) PCT No.: PCT/EP2023/068960

§ 371 Date: Jan. 17, 2025

Related U.S. Application Data
(60) Provisional application No. 63/390,988, filed on Jul. 21, 2022.

(51) Int. Cl. G16H 40/40; G16H 40/67

(52) U.S. Cl. CPC G16H 40/40; G16H 40/67

(57) ABSTRACT

A database stores summaries of historical service cases. A request for assistance is received for a current service case for a medical device. A context of the current service case is identified based at least on the received request. Candidate filter value combinations are generated for a plurality of filters for use in searching the summaries of historical service cases. The candidate filter combinations are generated based on the identified context of the current service case. The candidate filter value combinations are simulated by filtering the database using the candidate filter combinations. A recommended combination of filter values or a recommended service action is identified based on the simulating. On a display device of an electronic processing device operable by a service engineer (SE), the recommended combination of filter values or recommended service action is output.

### FIELD

The following relates generally to the medical imaging arts, medical imaging device maintenance arts, medical imaging device diagnostic procedure arts, and related arts.

### BACKGROUND

A medical imaging system or other medical devices may occasionally malfunction, and thus fail to work properly. The malfunction is recognized by an operator of the medical device based on observed symptoms, such as failure of the medical device to operate, unsatisfactory output of the medical device (e.g., a patient monitor unable to read a vital sign sensor, a medical imaging device producing low-quality images, or so forth), and/or by an error code produced by the medical device, or based on other symptoms. Depending on the malfunction, the problem can sometimes be resolved remotely, by a remote service engineer (RSE). During diagnosis, the RSE can interact with several tools that can aid during system troubleshooting, and identify the best course of action to resolve the problem. For example, the RSE may decide to perform several actions, such as searching through historical service records to find similar service cases, and identify how they were resolved, searching through technical documentation for possible root causes and potential solutions, inspecting the log files of the medical imaging system, to identify relevant diagnostic information such as error messages, contacting the customer to get a description of the problem, and so forth.

If the malfunction cannot be handled remotely, then the remote service engineer can issue a request for a Field Service Engineer (FSE) to repair the medical device on-site.

Depending on the stage of the diagnostic process, RSEs and FSEs can use various tools for assistance. Examples of such tools include expert diagnostic systems, search engines over knowledge bases (such as historical service records), tools to analyze log data from devices etc. The use of the tools, and how they are configured by RSEs depend on several factors, including, for example, obtained diagnostic information, a service contract of the customer, experience and skills of the service engineer (SE), availability of parts and engineers for maintenance, and so forth.

RSE's may not be able to correctly judge which option to select in each of the diagnostic tools. This can be because of their lack of experience, the complexity of the available choices, or the number of available options to choose from.

For example, engineers can use a search engine to find similar service records from the past, and to get recommendations for which service action to take. In this tool, they can configure filters such as product group, malfunction area, case priority, case country, case modality etc., but they can also specify different search terms, which influence both the search results and the recommended service actions. Search results can contain a few ten thousand cases. Incorrectly provided options may result in sub-optimal results, such as wrong or more expensive service actions than needed in the recommended actions.

Consequently, engineers sometimes perform multiple searches to reach a higher chance of selecting the right service actions. However this method of performing multiple searches is arduous and prone to human error. Moreover, the engineer may focus on technical aspects such as identifying possible service actions to attempt, but fail to recognize other important but less apparent information such as noticing how frequently (or infrequently) a given service action actually resolved a problem in the historical cases, and associated costs in terms of parts and time.

The following discloses certain improvements to overcome these problems and others.

### SUMMARY

In some embodiments disclosed herein, a non-transitory computer readable medium stores a database storing summaries of historical service cases, and instructions executable by at least one electronic processor to perform a method for assisting in resolving a current service case for a medical device. The method includes: receiving a request for assistance for the current service case for the medical device; identifying a context of the current service case based at least on the received request; generating candidate filter value combinations for a plurality of filters for use in searching the summaries of historical service cases, the candidate filter combinations being generated based on the identified context of the current service case; simulating the candidate filter value combinations by filtering the database using the candidate filter combinations; identifying a recommended combination of filter values or a recommended service action based on the simulating; and outputting, on a display device of an electronic processing device operable by a service engineer (SE), the recommended combination of filter values or recommended service action.

In some embodiments disclosed herein, a medical apparatus is disclosed, including a medical device and an electronic processing device comprising at least one electronic processor. The electronic processor is programmed to: identify a context for a current service case for a medical device; generate candidate filter value combinations based on the identified context of the current service case; simulate the candidate filter value combinations to identify a recommended combination of filter values; and apply the recommended combination of filter values to historical service cases related to the current service case.

In some embodiments disclosed herein, a method of recommending one or more filters for a search query for a current service case is disclosed. The method includes: identifying a context for a current service case for a medical device; generating candidate filter value combinations based on the identified context of the current service case; simulating the candidate filter value combinations to identify a recommended combination of filter values; applying the recommended combination of filter values to retrieve historical service cases related to the current service case from a database; and determining a recommended service action to resolve the current service case based on the retrieved historical service cases.

One advantage resides in recommending diagnostic tools to assist an RSE in performing a service case.

Another advantage resides in filtering historical service actions based on a search query from an RSE.

Another advantage resides in reducing a number of searches performed by a RSE to perform a service case.

Another advantage resides in improving the accuracy of search queries for current service cases.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates a medical device servicing apparatus in accordance with the present disclosure.

FIGS. 2 and 3 diagrammatically illustrate two embodiments of a medical device servicing method using the apparatus of FIG. 1.

FIG. 4 diagrammatically illustrates an embodiment of a user interface (UI) output of the method of FIG. 3.

### DETAILED DESCRIPTION

In surveys of users, newer service engineers (SEs) reported having difficulty navigating a system including historical service cases database analysis software to locate relevant historical cases for resolving current service cases for medical imaging systems. However, more surprisingly, even experienced RSEs reported having some difficulty navigating the system. In particular, they had difficulty selecting appropriate filters for selecting a few most relevant cases out of the tens of thousands or more historical service cases in the database.

In view of this, the following discloses an automated assistive method that provides a recommended set of filters for use in the system. In a variant embodiment, the system autonomously applies the recommended set of filters and then analyzes the returned cases to generate an actionable recommendation such as a recommended part replacement.

The method identifies a context of the current case. As the database analysis software logs events including entry of information such as an error code, user-provided problem description, or RSE summary, the context can be directly derived from the event log. These information are also used to generate candidate problem-specific search terms (e.g., the error code and/or keywords extracted from the problem description or summary).

The method also determines a performance indicator that can be used as a metric for assessing the “quality” of the historical cases returned using a given set of filters. The performance indicator may be based on the service contract and possibly other information, and may be a single indicator (e.g., cost or service time) or a weighted combination of indicators (e.g., W1*cost+W2*service time).

The method generates a matrix of possible filter value combinations. In some embodiments, there are filters for malfunction area, product group, and search terms. The possible filter values for a given filter are derived from the context using a lookup table (e.g., each error code can have an associated set of possible malfunction areas) or directly from the context (e.g., the search terms form possible freeform text filter values). In the case of product group, while a given current case will be for a single product, there may be a larger group of products for which relevant historical cases may be of value, for example if a whole product group uses a particular module of concern.

Each of these filters can (in general) assume a range of values, so that the matrix (even for this simplified example of only three filters) can include dozens or hundreds of candidate filter value combinations. Simulations are then run. A “simulation” in this context refers to applying a candidate set of filter values and evaluating the returned set of historical cases with respect to the performance indicator. Such a simulation is run for each candidate filter value combination of the matrix. Based on the simulations and performance indicator evaluations, a recommended combination of filter values is selected.

Finally, a user interface (UI) of the historical cases analysis tool is modified to present the recommended combination of filters. In some embodiments, this is presented as a proposal that the RSE can select or reject or modify (for example, by removing a recommended filter or modifying a recommended filter or adding a filter in addition to those recommended). In another approach, the recommended combination of filters is automatically applied. In either case, the returned cases may be analyzed to determine an actionable recommendation such as a part replacement recommendation, which is then presented via the UI.

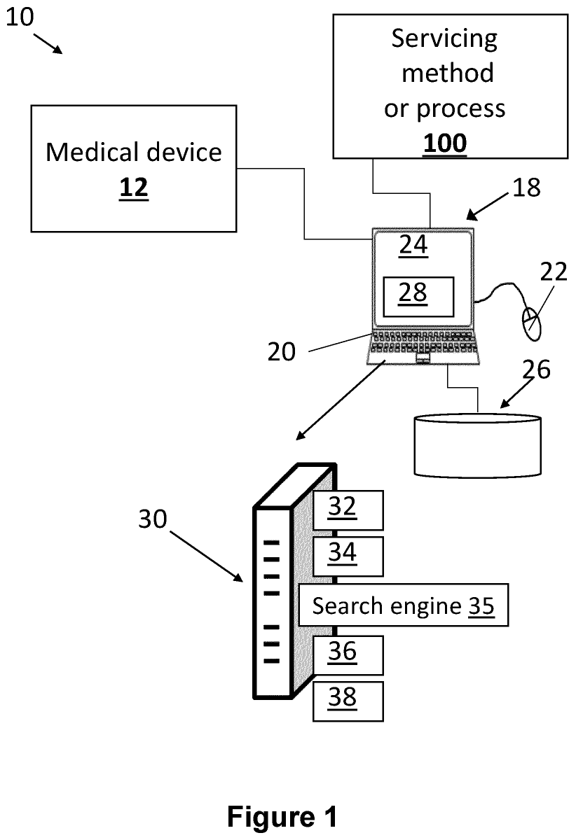

With reference to FIG. 1, an illustrative apparatus 10 for servicing a medical device 12 is shown. The medical device 12, for example, can comprise a medical imaging device 12 (also referred to as a medical device, an imaging device, imaging scanner, and variants thereof) can be a magnetic resonance imaging (MRI) scanner, a computed tomography (CT) scanner, a positron emission tomography (PET) scanner, a gamma camera for performing single-photon emission computed tomography (SPECT), an interventional radiology (IR) device, an X-ray device, an image-guided therapy (iGT) device, or so forth. Although shown in FIG. 1 as an imaging device, the medical device 12 can also be any other suitable medical device, such as a patient monitor, a radiation therapy device, a mechanical ventilator, and so forth.

An electronic processing device 18, such as a workstation computer, or more generally a computer, a smart device (e.g., a cellular telephone (“cell phone”), a smart tablet, and so forth), is operable by a service engineer (SE). The electronic processing device 18 may also include a server computer or a plurality of server computers, e.g., interconnected to form a server cluster, cloud computing resource, or so forth, to perform more complex computational tasks. The electronic processing device 18 includes typical components, such as an electronic processor 20 (e.g., a microprocessor), at least one user input device (e.g., a mouse, a keyboard, a trackball, and/or the like) 22, and a display device 24 (e.g., an LCD display, plasma display, cathode ray tube display, and/or so forth). In some embodiments, the display device 24 can be a separate component from the electronic processing device 18, or may include two or more display devices.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 28 for display on the display device 24.

The electronic processing device 18 is also in communication with a database 30 (shown in FIG. 1 as a server computer) that stores storing summaries 32 of historical service cases related to diagnostic procedures for a plurality of different medical devices (including the medical device 12 shown in FIG. 1). The summaries 32 of the historical service cases may include information such as one or more of: a description of the reason for each service call and/or the problem addressed in the service call (e.g., symptoms described by the customer, a problem summary written by the service engineer, automatic warnings, fault notifications or the like generated by the imaging device, and/or so forth), information on the medical imaging device being serviced in each service call (e.g., imaging modality, imaging device vendor, imaging device model, imaging device serial number, relevant imaging device configuration data, geographical location of the imaging device, customer/owner of the imaging device, et cetera), a list of service actions performed in each historical service case, a list of part numbers of parts that were replaced (if any) in each historical service case, maintenance time information (e.g. hours billed or recorded by the service engineer handling the historical service case), total cost billed to the customer (or incurred by the service provider, depending on the contractual arrangement) for each historical service case, and/or so forth. The database 30 also stores a plurality of filters 34 for use in a search query executed by a search engine 35 implemented by the electronic processing device 18 for performing the search query on the database 30. For example, in the case of a database of medical imaging system service cases, the plurality of filters may include filters such as a product group filter, a malfunction area filter, a case priority filter, a case country filter, a case modality filter (e.g., magnetic resonance (MR), computed tomography (CT), positron emission tomography (PET), et cetera), and so forth. More generally, each filter that is used can be set to a range of possible values, depending on the type of data being filtered by that filter. For example, the case country filter may be set to any country from which historical service cases are present in the database 30; the malfunction area filter can be set to any one or more of a set of predefined areas of the medical imaging system (e.g., in the case of MRI these might include the magnet, the patient support, the magnetic field gradient coils, or so forth, by way of nonlimiting illustrative example). The value of a filter may also be in the form of a set or other data structure, e.g. the value of the malfunction area in the MRI example might be set to “magnet OR magnetic field gradient coils” to enable returning historical cases in which the malfunction area was either the magnet or the magnetic field gradient coils. The filters may be presented to the SE (or other user) as drop-down selection graphical user interface (GUI) dialogs, GUI checkbox dialogs, or the like. The SE can then select filters to apply in order to cause the search engine 35 to retrieve, from the database 30, summaries of historical service cases that are similar to the current service case. Ideally, the set of values of the filters chosen by the SE cause the search engine to return summaries of a relatively small number of highly relevant historical service cases, which the SE can then review in order to identify one or a sequence of service actions that successfully resolved many of the similar historical service cases (and hence is likely to resolve the current service case). However, in practice it is often found that the SE is unable to formulate such as well-designed set of filter values. It will be appreciated that the number of summaries 32 of historical service cases stored in the database 30 may be large, e.g. tens of thousands of cases, hundreds of thousands of cases, or more, and a search using a given set of values for the plurality of filters 34 can in many cases return a few tens of thousands of historical service case summaries (or more). Such a large number of search result are of little value to the SE who will be unable to review this multiplicity of results. On the other hand, if the set of filter values produces a too-narrow search, then highly relevant summaries may be missed.

To address such problems, the apparatus 10 is configured as described herein to perform a method or process 100 of recommending a set of filter values for one or more filters 34 for a search query on the database for a current service case. The recommendation method or process 100 operates on context of the current service case determined from information such as the received request itself, and/or automated analysis of log data received from the medical imaging device 12. In one approach, candidate filter value combinations are generated based on the identified context of the current service case, each candidate filter value combination is simulated (for example, by applying it against the database 30 using the search engine 35 to determine how many results are returned and analyzing relevance of those results), and identifying a recommended combination of filter values based on the simulating. The identification can be based on criterion such as, by way of nonlimiting illustrative example, selecting a filter combination that returns a reasonable number of results with highest relevance. A “reasonable” number of results can be quantitatively defined, by way of nonlimiting illustrative example, as the number of returned historical case summaries being in a range Nₘᵢₙ to Nₘₐₓ where Nₘᵢₙ is set to one or to some small number of summaries easily consumed by the SE and Nₘₐₓ is set to a number of summaries that is the largest number of summaries the SE can be reasonably expected to consume (e.g. read/analyze) as part of the resolution of the current service case. In some embodiments, Nₘₐₓ (and possibly also Nₘᵢₙ) may be user-configurable parameters that can be set by the SE. The non-transitory storage medium 26 stores instructions which are readable and executable by the at least one electronic processor 20 to perform disclosed operations including performing the servicing method or process 100. In some examples, the method 100 may be performed at least in part by cloud processing.

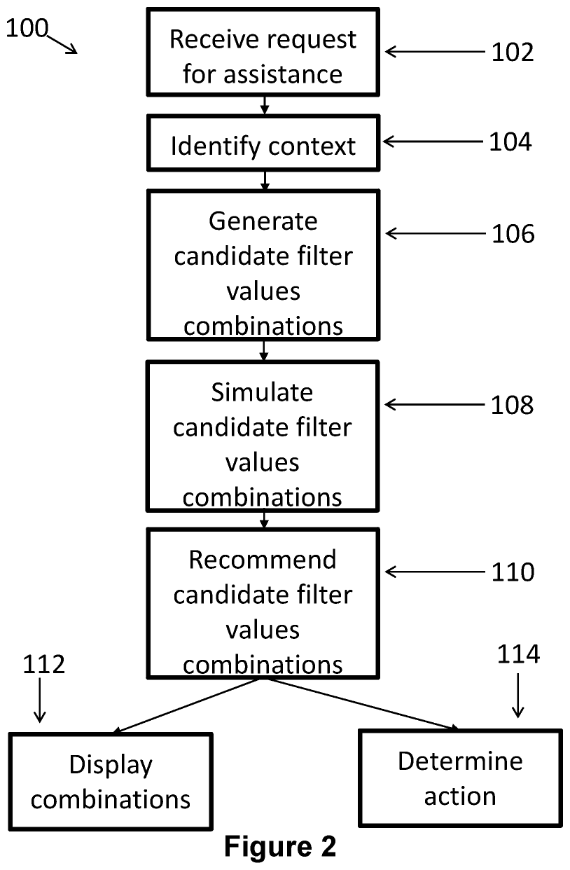

With reference to FIG. 2, and with continuing reference to FIG. 1, an illustrative embodiment of an instance of the method 100 is diagrammatically shown as a flowchart. At an operation 102, a request for assistance for a current service case for the medical device 12 is received. In some examples, the received request comprises error codes generated by the medical device 12, user-provided problem descriptions, historical search results or a summary from the RSE.

At an operation 104, a context of the current service case is identified based at least on the received request. For example, the received request may be entered as natural language text (e.g., by typing or by automatic transcription of a verbally stated request captured by a microphone) and the operation 104 may include extracting key words or phrases from the request that provide context, such as the imaging modality (MRI, CT, et cetera), keywords in a description of the problem (e.g., mentions of components that may indicate the malfunction area, such as “patient support” possibly indicative of a problem in the patient support malfunction area, or “magnetic field” possibly indicative of a problem in the magnet malfunction area or magnetic field gradient coils malfunction area), identification of the country in which the medical imaging device is located, and/or so forth. In some embodiments, the identifying operation 104 includes analyzing log data received from the medical device 12, and identifying the context further based on the analyzed log data. For example, the log data may be analyzed to identify recent automatically generated alarms, alerts, or other indications of a malfunction contained in the log data. As another example, the log data may be analyzed to identify specific types of imaging examinations during which such alarms, alerts, or the like occurred. Based on such information, the operation 104 can determine a context, such as (by way of nonlimiting illustrative example): MRI, magnet or gradient coil malfunction, alarm < . . . >, imaging sequences < . . . >. (Where in this example, < . . . > indicates specific alarms and imaging sequences during which such alarms were triggered, as extracted from the log data).

At an operation 106, candidate filter value combinations are generated based on the identified context of the current service case. For example, the candidate filter value combinations include filter values for filters including a malfunction area filter, a product group filter, a search terms filter, filters from different remote diagnosis tools, and so forth.

In one embodiment, the generating operation 106 includes generating at least one candidate problem-specific search term from the identified context of the current service case. The at least one problem-specific search term forms at least one candidate free-form text filter value for the candidate filter value combinations.

In another embodiment, the generating operation 106 includes generating a matrix of candidate filter value combinations deriving the candidate filter value combinations based on the identified context. In the above example, the matrix of candidate filter value combinations may include combinations with the value for the imaging modality filter set to “MRI” along with various combinations of “magnet” and/or “magnetic field gradient coils” as the malfunction area filter values, and so forth. Some contextual information may be incorrect. For example, the user may have mentioned the magnet in the received request, but this may have been extraneous language unrelated to the root cause of the problem, or the SE may be mistaken in guessing that the root cause relates to the magnet; similarly some automatically generated alerts or alarms may be unrelated to the root cause. Hence, in some embodiments the operation 106 may generate some candidate filter value combinations that deviate from those directly derivable from the context. For example, one or some the candidate filter value combinations might include “magnet coolant” as a malfunction area that is searched; while one or more other candidate filter value combinations might include “RF coil” as the malfunction area in spite of the context not mentioning the RF coil(s). In another embodiment, the generating operation 106 includes deriving the candidate filter value combinations based on the identified context using a lookup table 36 implemented in the database 30.

At an operation 108, the candidate filter value combinations are simulated by filtering the database 30 using the candidate filter combinations. For example, the operation 108 may entail applying each candidate filter value combination against the database 30 using the search engine 35 to retrieve the matching historical service case summaries, and these results are analyzed to determine as to the number of summaries returned and relevance metrics for the returned results. The relevance metrics could include metrics such as a frequency of context keywords contained in the summaries, time-to-resolution of the historical service case, total cost of resolution (quantified for a given historical case as a sum of the monetary-equivalent value of SE time expended and cost of replacement parts if any, for example), and/or so forth. As recognized herein, this processing entailing applying each candidate filter value combination using the search engine 35 can be computationally efficient, since the returned content may only be textual summaries of the returned historical service cases, for example. Optionally, the search engine 35 may be configured to make the operation 108 more efficient by, for example, setting query result configuration settings to minimize the amount of content returned. For example, if the search engine 35 is configurable to return only summaries or to return both summaries and additional information then the “only summaries” configuration can be selected when querying the candidate filter value combinations against the database 30.

At an operation 110, a recommended combination of filter values is identified from the candidate filter value combinations based on the simulating. To do so, the identifying operation 110 includes is based at least in part on values of a performance indicator 38 for assessing a quality of historical service cases applied to historical service cases returned by the simulating. The performance indicator 38 for each candidate combination of filter values may be based on (by way of nonlimiting illustration) whether the number of returned historical case summaries is in the reasonable range [Nₘᵢₙ, Nₘₐₓ] and an aggregation of the relevance metrics for each candidate combination. In one example, the performance indicator 38 is based (or further based) on a service contract for the medical device 12. In another example, the performance indicator 38 comprises at least one of a cost indicator and/or a service time indicator.

In one embodiment, at an operation 112, the recommended combination of filter values is output on the display device 24 of the electronic processing device 18. For example, the recommended combination of filter values is displayed on the display device 24, and an input is received (via the at least one user input device 22) from the RSE indicative of an acceptance or rejection of the recommended combination of filter values. Optionally, the RSE response may further include modifying the recommended combination of filter values, for example, by adding additional filter values, removing one or more of the recommended filter values, and/or modifying one or more of the recommended filter values.

In another embodiment, at an operation 114, the recommended combination of filter values is applied to relevant historical service cases 32 related to the current service case. The relevant historical service cases related to the current service case 32 are analyzed, and an actionable recommendation to resolve the current service case is determined.

### EXAMPLE

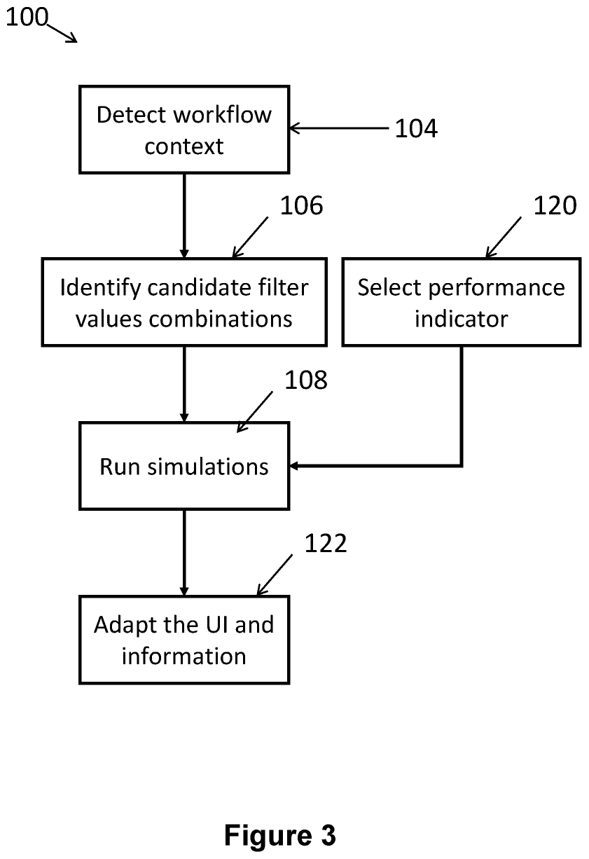

With reference to FIG. 3, a variant of the embodiment of FIG. 2 is used in the following examples. In this variant, rather than returning a recommended filter values combination, the output is one or more recommended service actions extracted by actually applying the best filter values combination or combinations. As before, a workflow context detection unit (implemented in the electronic processing device 18) is configured to perform the workflow context detection 104 to detect where the user (e.g., RSE A received this case for the first time) is in this case's workflow, as well as the context of the customer (e.g., Customer X persistently received “not found” after a few times rebooting). In the simplest case, the context can be extracted from the service log or from the call center (e.g. problem description), using Natural Language Processing (NLP) technologies. In case more information is requested, such as how many searches the user has performed, which filters the user has applied, the additional information can be extracted from the logging data of the remote diagnosis tool.

A performance indicator finding unit (implemented in the electronic processing device 18) is configured to perform an operation 120 to find a performance indicator from hospital contract and its details. The performance indicator may be chosen based on the service contract with the hospital, since different contract terms may change the costs to the service provider of service time versus replacement components and other costs. As an example, for strict contracts with hospitals (e.g., high penalties to the service provider for downtime of an imaging device), the performance indicator 38 can be total maintenance time (or machine down time). For service contracts with pay-per-service billing, the performance indicator can be the cost of replacement parts. To find the performance indicator 38, the look up table 36 as shown in Table 1 can be used, in which the input to the look-up table 36 is the type of service contract and the output is the performance indicator 38. The performance indicator 38 may be based on other metrics, such as availability of parts, availability of service engineers to perform the maintenance, and/or so forth. In some embodiments, the performance indicator 38 may be selected or modified based on current circumstances. For example, during a major holiday when many service personnel may be unavailable, the performance indicator 38 may be modified to more heavily weight availability of service personnel, so that a historical case summary that resolved the case without requiring dispatch of a field service engineer to the site might be assigned higher performance during such holiday intervals. To facilitate such modification, the performance indicator could in some embodiments be implemented as a weighted sum of constituent performance indicators (maintenance time, parts cost, et cetera).

TABLE 1
An example of a lookup table to find a performance indicator

| Type of contract | Performance indicator |
|---|---|
| Strict with penalty | Maintenance time |
| Pay per service | Cost |

The operation 106 of identifying the candidate filter value combinations is also performed. A set (or combination) of actions unit (implemented in the electronic processing device 18) is configured to identify a set of actions (with the diagnostic tools) or a set of combinations of actions (i.e., filter values) the user can use to filter the database 30 based on the workflow context. For instance, when a user just performed a user message search on historical cases, the next available actions to further refine the search results are listed in set 1 of actions listed in Table 2. The actions become parameters (i.e. filter values) for the simulation. The way to identify the set of actions can be from a maintained lookup table (such as Table 2), or automatically extracted from the available diagnostic tools.

TABLE 2
An example of a set of combination of actions identified.

| | | | Number of |
|---|---|---|---|
| Set of | Filters for | | possible filter |
| actions | the context | Possible filter values | values |
| 1 | Malfunction | None, No parts, computer, | 4 |
| | area filter | coil | |
| 2 | Product group | None, serie 7, serie 8, serie 9 | 4 |
| | filter | | |
| 3 | Search terms | Error message, problem | 3 |
| | | description, Engineer | |
| | | summary | |
| 4 | Service action | Cost, availability, | 3 |
| | optimization | maintenance time | |
| | target | | |

TABLE 3
An example of performance indicators, or
optimization targets for the simulations.

| Number | Optimization target |
|---|---|
| 1 | Cost |
| 2 | Availability |
| 3 | Maintenance time |

Next, the operation 108 is performed to run the simulations. The simulations in this illustrative example can include three operations. First, a search query is executed in the search engine 35 for service records using each candidate filter values combination, and a set of service cases as search results is output. Next, the search results are processed by a service action recommendation engine, and a set of service actions is output. The processing may include, for example, analyzing the historical case summaries returned by the searches to extract the service actions that were performed in the historical cases. This can, for example, entail performing natural language processing on freeform content of the returned historical service case summaries to identify the performed service actions, and/or by detecting parts numbers included in the summaries thereby indicating part replacement service actions, and/or so forth. Finally, the quality of the recommended service actions is evaluated against a set of performance indicators (i.e. metrics, such as in Table 3). The performance indicators may for example be selected in the operation 120 based on the service contract as previously discussed with reference to Table 1, and/or based on other information. The output of this evaluation of each service action is a number between 0 and 1 indicating the simulation quality (0—worst, 1—best possible quality) for that service action. For example, if the parts cost is chosen in operation 120 as the performance indicator, than the parts cost can be computed for each historical service case that includes that service action. (Optionally, the historical parts costs can be adjusted to reflect the current parts prices). If the maintenance time is chosen as the performance indicator, then the average maintenance time is determined for the historical service cases that include that service action. These are merely nonlimiting illustrative examples. In addition to determining the performance indicator for each service action, the recommended service actions are evaluated in terms of likelihood of resolving the malfunction, further denoted as Success Chance. This can be based on the fraction of historical service cases in which a given service action is performed that were resolved by that service action. In this setting, the simulations parameters can influence either the search engine 35, through the specification of the search query, or the service action recommendation engine, by specifying the optimization criteria.

To further illustrate, for the example from Table 2 and performance indicators from Table 3, there are 4 different sets of options to be investigated, resulting in 144 different configurations for simulation (i.e., referring to the example of Table 2, there are 4 possible filter values for the malfunction area filter times four possible filter values for the product group filter times three possible filter values for the search terms filter times three possible filter values for the service action optimization target filter, i.e. 4×4×3×3=144 candidate filter value combinations). Each of these 144 candidate filter value combinations is executed by the simulation engine (e.g., using the search engine 35), and the best results are shown to the user (e.g. after a pareto analysis has been performed according to one of the optimization criteria). An example of the results is shown in Table 3.

TABLE 4
An example of a result of the simulation.

| | | | | Best | |
|---|---|---|---|---|---|
| | | | | performance | |
| | Malfunction | Product | Search | according to | Success |
| Setting | area | group | terms | indicator | Chance |
| 1 | none | serie 8 | Error | Cost | 0.5 |
| | | | message | | |
| 2 | Computer A | none | problem | Availability | 0.7 |
| | | | description | | |
| 3 | Computer B | serie 9 | problem | maintenance | 0.7 |
| | | | description | time | |

According to these results, the service action with the least cost has a 0.5 chance of success, and can be obtained by searching for the error message found by the service engineer in the previous steps, and setting only the product group filter to ‘serie 8’. The service action optimized for availability and repair time is identical, and has a 0.7 chance of success. It can be obtained by searching for the user provided description, using the malfunction area filter set to ‘Computer A.’

A UI and information adaptation unit (implemented in the electronic processing device 18) is configured to adjust the best search setting, and adapts the UI and information in an operation 122 of FIG. 3. For example, a private hospital with a new contract received error “not found” on an iXR. The error kept appearing even after rebooting the system 5 times. The workflow context detection unit detects that the RSE has performed a search on “not found” (without any filters), the identify a set (or combination) of actions unit finds the possible set of actions as listed in setting 1 in Table 2, the performance indicator finding unit finds that the performance indicator for this hospital is Chance of Success based on one of a cost, availability, or maintenance time performance indicator.

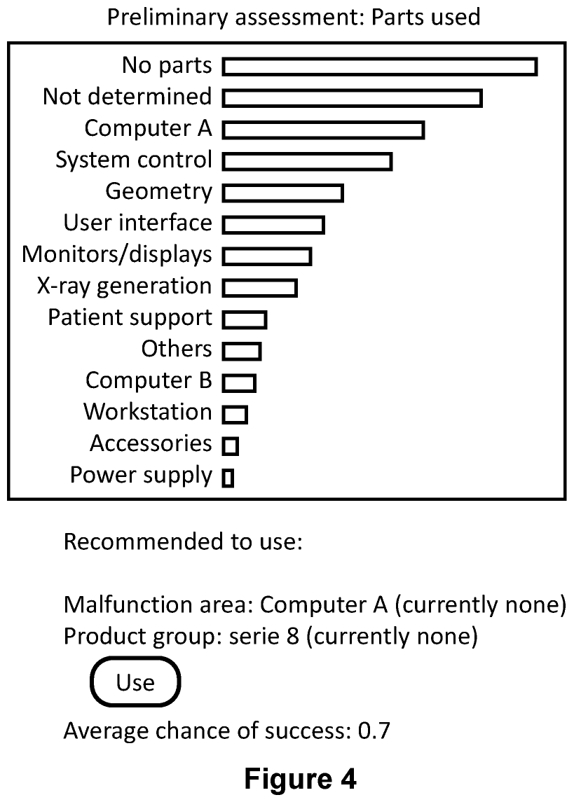

With reference to FIG. 4, an illustrative example of an adapted UI and information output by the operation 122 of FIG. 3 is shown. This example is for a service case for a medical X-ray imaging system. After running the simulation, the set of actions (i.e. filter values) with the best chance of success is setting the malfunction area filter to “Computer A” and the product group filter to “serie 8”. The adapted UI recommends searching the database 30 using “Computer A” as the value for the malfunction area filter, and “serie 8” as the value for the Product group filter. Based on the success rate from the simulations, the average chance of success in resolving the case using these filter values is estimated at 70% (i.e., “Average chance of success: 0.7” in FIG. 4). Note that while FIG. 4 shows the “Preliminary assessment” as a bar graph, other graphical formats such as pie chart could be used, and/or numerical values.

Alternatively, the UI and information adaptation unit is not limited to showing the best set of actions as a final recommendation, it can also show the top ones or the whole list. In case the current set of actions is quantified as below the mean of the performance indicator, the unit can show the user such guidance in a step-by-step fashion.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing:
a database storing summaries of historical service cases; and
instructions executable by at least one electronic processor to perform a method for assisting in resolving a current service case for a medical device, the method including:
receiving a request for assistance for the current service case for the medical device;
identifying a context of the current service case based at least on the received request;
generating candidate filter value combinations for a plurality of filters for use in searching the summaries of historical service cases, the candidate filter combinations being generated based on the identified context of the current service case;
simulating the candidate filter value combinations by filtering the database using the candidate filter combinations;
identifying a recommended combination of filter values or a recommended service action based on the simulating; and
outputting, on a display device of an electronic processing device operable by a service engineer (SE), the recommended combination of filter values or recommended service action.

2. The non-transitory computer readable medium of claim 1, wherein identifying a context of the current service case further includes:
analyzing log data received from the medical device; and
identifying the context further based on the analyzed log data.

3. The non-transitory computer readable medium of claim 1, wherein the received request comprises error codes generated by the medical device, user-provided problem descriptions, historical search results, or a summary from the SE.

4. The non-transitory computer readable medium of claim 2, wherein the generating of the candidate filter value combinations includes:
generating at least one candidate problem-specific search term from the identified context of the current service case, the at least one problem-specific search term forming at least one candidate free-form text filter value for the candidate filter value combinations.

5. The non-transitory computer readable medium of claim 1, wherein the generating of the candidate filter value combinations includes:
generating a matrix of candidate filter value combinations.

6. The non-transitory computer readable medium of claim 1, wherein the generating of the candidate filter value combinations includes:
deriving the candidate filter value combinations based on the identified context using a lookup table.

7. The non-transitory computer readable medium of claim 5, wherein the candidate filter value combinations include filter values for filters including a malfunction area filter, a product group filter, a search terms filter, and filters from different remote diagnosis tools.

8. The non-transitory computer readable medium of claim 1, wherein the identifying of the recommended combination of filter values or recommended service action from the candidate filter value combinations is based at least in part on values of a performance indicator for assessing a quality of historical service cases applied to historical service cases returned by the simulating.

9. The non-transitory computer readable medium of claim 8, wherein the performance indicator is based on a service contract for the medical device.

10. The non-transitory computer readable medium of claim 8, wherein the performance indicator comprises at least one of a cost indicator and/or a service time indicator.

11. The non-transitory computer readable medium of claim 1, wherein the recommended combination of filter values or recommended service action is a recommended combination of filter values, and the outputting, on the display device, of the recommended combination of filter values includes:
displaying the recommended combination of filter values; and
receiving, via at least one user input device, an input from the SE indicative of an acceptance or rejection of the recommended combination of filter values.

12. The non-transitory computer readable medium of claim 1, wherein the recommended combination of filter values or recommended service action is a recommended combination of filter values, and the method further comprises:
applying the recommended combination of filter values to retrieve historical service cases from the database.

13. The non-transitory computer readable medium of claim 1, wherein the recommended combination of filter values or recommended service action is a recommended service action, and the method further includes:
analyzing the historical service cases related to the current service case; and
determining the recommended service action to resolve the current service case based on the analysis.

14. A medical apparatus, comprising:
a medical device; and
an electronic processing device comprising at least one electronic processor programmed to:
identify a context for a current service case for a medical device;
generate candidate filter value combinations based on the identified context of the current service case;
simulate the candidate filter value combinations to identify a recommended combination of filter values; and
apply the recommended combination of filter values to historical service cases related to the current service case.

15. The medical apparatus of claim 16, wherein the generating of the candidate filter value combinations includes:
generating a matrix of candidate filter value combinations.

16. The medical apparatus of claim 15, wherein the generating of the matrix includes:
deriving the candidate filter value combinations based on the identified context.

17. The medical apparatus of claim 14, wherein simulating the candidate filter value combinations to identify a recommended combination of filter values includes:
applying a candidate set of filter values to the candidate filter value combinations to identify potentially relevant historical service cases related to the current service case; and
evaluating the identified potentially relevant historical service cases based on the performance metric.

18. The medical apparatus of claim 14, wherein identifying a context of the current service case further includes:
analyzing log data received from the medical device; and
identifying the context further based on the analyzed log data.

19. A method of recommending one or more filters for a search query for a current service case, the method including:
identifying a context for a current service case for a medical device;
generating candidate filter value combinations based on the identified context of the current service case;
simulating the candidate filter value combinations to identify a recommended combination of filter values;
applying the recommended combination of filter values to retrieve historical service cases related to the current service case from a database; and
determining a recommended service action to resolve the current service case based on the retrieved historical service cases.

20. The method of claim 19, further including:
identifying the recommended combination of filter values from the candidate filter value combinations based at least in part on values of a performance indicator for assessing a quality of historical service cases applied to historical service cases returned by the simulating.
